(19) United States
(12) Patent Application Publication
(10) Pub. No.: US 2014/0012082 A1
(43) Pub. Date: Jan. 9, 2014

(54) ENDOSCOPE AND ENDOSCOPE SYSTEM

(71) Applicant: SAMSUNG ELECTRONICS CO., LTD., Suwon-si (KR)

(72) Inventors: Joon-kee CHO, Yongin-si (KR); Hyung-joo KIM, Seongnam-si (KR); Jong-hwa WON, Seoul (KR)

(73) Assignee: SAMSUNG ELECTRONICS CO., LTD., Suwon-si (KR)

(21) Appl. No.: 13/863,639

(22) Filed: Apr. 16, 2013

(30) Foreign Application Priority Data
Jul. 3, 2012 (KR) 10-2012-0072405

(51) Int. Cl. A61B 1/00

(52) U.S. Cl. CPC A61B 1/00193
USPC 600/111; 600/109

(57) ABSTRACT

An endoscope includes a first lens unit that collects light corresponding to an object image which is an image including depth information of an object; a second lens unit that collects light corresponding to a surrounding tissue image which is an image of a surrounding tissue of the object; a switching unit that alternately applies light received from the first lens unit and the second lens unit to a photographing unit; and a photographing unit that alternately captures the light from the switching unit.

### CROSS-REFERENCE TO RELATED APPLICATIONS

This application claims the priority benefit of Korean Patent Application No. 10-2012-0072405, filed on Jul. 3, 2012, in the Korean Intellectual Property Office, the disclosure of which is incorporated herein in its entirety by reference.

### BACKGROUND

1. Field

The following description relates to an endoscope that provides an image of an object and a surrounding tissue, and an endoscope system.

2. Description of the Related Art

An endoscope is a medical instrument that is directly inserted into a body whose lesion may not be visible and shows an interior of an organ or a body cavity without an operation or an incision. At an initial stage, an endoscope was simply used to observe an organ or the like in a body cavity by inserting a narrow and long insertion unit into the body cavity. As the image processing technology has been developed, each part in a body cavity has been photographed by using a black-and-white camera and then a lesion in each part has been closely observed by using a captured image. Such a simple black-and-white camera has been replaced with a high-resolution color imaging apparatus, and thus a lesion has been more closely observed by using the high-resolution color imaging apparatus. Also, a chromo endoscope that dyes a surface of a body cavity with a specific pigment according to a lesion to be detected and captures an image has been used.

Meanwhile, an endoscope which provided only a black-and-white image in the past has been developed to a level where it may provide a high-resolution color image and a narrow band image. The development of an endoscope is closely tied to the ability to accurately detect a lesion. Because conventional endoscopes provide only a two-dimensional (2D) image, it is difficult to accurately detect a lesion.

Also, because an endoscope photographs only an object to be operated, it is difficult to know whether the endoscope has damaged another tissue around the object. Accordingly, an endoscope that may capture images of an object and a surrounding tissue has been studied.

### SUMMARY

Provided are endoscopes and endoscope systems which may obtain depth information of an object and provide both an image including the depth information of the object and an image of a surrounding tissue of the object.

Additional aspects will be set forth in part in the description which follows and, in part, will be apparent from the description, or may be learned by practice of the presented embodiments.

According to an aspect of the present disclosure, an endoscope includes a first lens unit that collects light corresponding to an object image, which is an image including depth information of an object; a second lens unit that collects light corresponding to a surrounding tissue image, which is an image of a surrounding tissue of the object; a switching unit that alternately applies light received from the first lens unit and the second lens unit to a photographing unit; and a photographing unit that alternately captures the light from the switching unit.

A viewing field of the second lens unit may be greater than a viewing field of the first lens unit.

A viewing area of the second lens unit and a viewing area of the first lens unit may partially overlap each other.

The switching unit may apply light received from the first lens unit to the photographing unit during a first time interval, and apply light received from the second lens unit to the photographing unit during a second time interval.

The switching unit may include a first shutter unit that opens and closes the first lens unit; a second shutter unit that opens and closes the second lens unit; and a reflecting unit that transmits part of light incident from the first and second lens units and reflects others.

The reflecting unit may reflect light incident from the first lens unit to the photographing unit, and transmit light incident from the second lens unit to the photographing unit.

The reflecting unit may include a half-mirror.

The switching unit may be an optical modulator that reflects light incident from the first lens unit, and transmits light incident from the second lens unit.

The optical modulator may alternately reflect and transmit light incident according to an electrical control signal.

The surrounding tissue image may be a two-dimensional (2D) image.

The endoscope may further include a first insertion unit that may be adapted to be inserted into a body of a person to be diagnosed; and a second insertion unit that is disposed on a front end of the first insertion unit and on which the photographing unit, the first lens unit, the second lens unit, and the switching unit are provided.

The second insertion unit may be pivotable about the front end of the first insertion unit.

The second lens unit may be disposed to face in a longitudinal direction of the first insertion unit before the second insertion unit is inserted into the body of the person to be diagnosed, and may be disposed to face perpendicular to the longitudinal direction of the first insertion unit after the second insertion unit is inserted into the body of the person to be diagnosed.

The first lens unit may be disposed to face perpendicular to a longitudinal direction of the first insertion unit before the second insertion unit is inserted into the body of the person to be diagnosed, and may be disposed to face in the longitudinal direction of the first insertion unit after the second insertion unit is inserted into the body of the person to be diagnosed.

The photographing unit may include: a first photographing unit that generates a left-eye image of the object; and a second photographing unit that generates a right-eye image of the object.

The first lens unit may include: a first lens that generates the left-eye image of the object; and a second lens that generates the right-eye image of the object.

The second lens unit may include: a third lens that generates a left surrounding tissue image of the object; and a fourth lens that generates a right surrounding tissue image of the object.

The first photographing unit may capture the left surrounding tissue image and the second photographing unit may capture the right surrounding tissue image of the object.

According to an aspect of the present disclosure, an endoscope system includes: the endoscope; and a processor that generates a panoramic image by combining the object image and the surrounding tissue image.

The endoscope system may further include a display device that displays the panoramic image.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram illustrating an endoscope system according to an embodiment of the present disclosure;

FIG. 2 is a block diagram illustrating an endoscope of the endoscope system of FIG. 1;

FIG. 3 is a perspective view illustrating the endoscope according to an embodiment of the present disclosure;

FIG. 4 is a cross-sectional view illustrating an optical arrangement when an endoscope photographs an object and a surrounding tissue of the object, according to an embodiment of the present disclosure;

FIG. 5A is a cross-sectional view illustrating an optical path when the endoscope photographs the object;

FIG. 5B is a cross-sectional view illustrating an optical path when the endoscope photographs the surrounding tissue of the object;

FIG. 6 is a cross-sectional view illustrating an optical arrangement of the endoscope, according to another embodiment of the present disclosure; and

FIG. 7 is a block diagram illustrating a processor according to an embodiment of the present disclosure.

### DETAILED DESCRIPTION

The present invention will now be described more fully with reference to the accompanying drawings, in which exemplary embodiments of the invention are shown. Widths and thicknesses of layers or regions in the accompanying drawings may be exaggerated for clarity. The same reference numerals denote the same elements throughout.

Embodiments of the present disclosure will now be explained in detail with reference to the accompanying drawings.

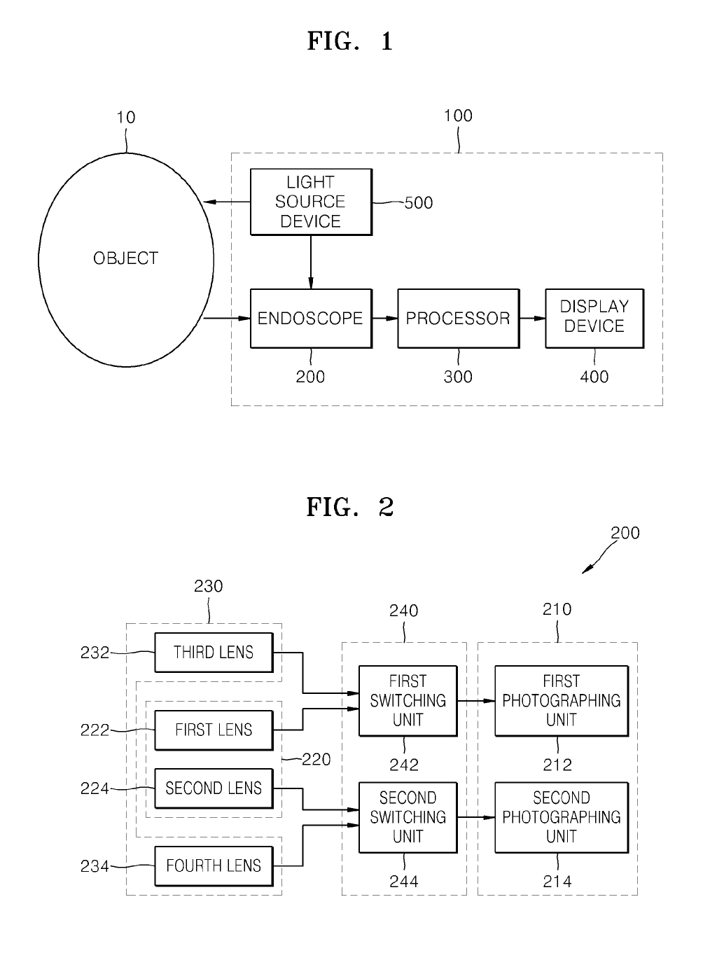

FIG. 1 is a block diagram illustrating an endoscope system 100 according to an embodiment of the present disclosure. FIG. 2 is a block diagram illustrating an endoscope 200 of the endoscope system 100 of FIG. 1.

Referring to FIG. 1, the endoscope system 100 includes the endoscope 200, a processor 300, a display device 400, and a light source device 500. The endoscope 200 may be connected to the processor 300 in a wired or wireless manner. The endoscope 200 may be connected to the light source device 500 via an optical fiber or the light source device 500 may be disposed in the endoscope 200. The processor 300 and the display device 400 may be disposed in a single housing.

The endoscope 200 is a device that is adapted to be inserted into a body of a person to be diagnosed and photographs an organ or a body cavity as an object 10. In detail, the endoscope 200 may include a photographing unit 210 that alternately photographs the object 10 and a surrounding tissue of the object 10, a first lens unit 220 that collects light corresponding to an image including depth information of the object 10 (hereinafter, referred to as an ‘object image’), a second lens unit 230 that collects light corresponding to an image of the surrounding tissue of the object 10 (hereinafter, referred to as a ‘surrounding tissue image’), and a switching unit 240 that applies light received from the first lens unit 220 or the second lens unit 230 to the photographing unit 210.

The photographing unit 210 photographs the object 10 in order to obtain the object image or photographs the surrounding tissue of the object 10 in order to obtain the surrounding tissue image. For example, the photographing unit 210 may include a first photographing unit 212 that captures a left-eye image of the object 10 and a second photographing unit 214 that captures a right-eye image of the object 10. The first photographing unit 212 may capture a left surrounding tissue image of the object 10 and the second photographing unit 214 may capture a right surrounding tissue image of the object 10. Each of the first and second photographing units 212 and 214 may include a complementary metal oxide semiconductor (CMOS) image sensor or a charge-coupled device (CCD) image sensor.

The first lens unit 220 may include first and second lenses 222 and 224 on which light reflected by the object 10 is incident in order to generate the object image. The first lens 222 may be a lens for generating the left-eye image of the object 10 and the second lens 224 may be a lens for generating the right-eye image of the object 10. The first and second lenses 222 and 224 may be disposed in the endoscope 200 to be spaced apart from each other in order to obtain the depth information of the object 10 through the first and second lenses 222 and 224, and may be disposed to face the object 10 when inserted into the body of the person to be diagnosed. Because the first and second lenses 222 and 224 are used to more precisely obtain a picture of the object 10 to be operated, viewing fields of the first and second lenses 222 and 224 may be narrow. For example, the viewing fields of the first and second lenses 222 and 224 may be approximately 70 degrees.

The second lens unit 230 may include third and fourth lenses 232 and 234 on which light reflected by the surrounding tissue of the object 10 is incident in order to generate the surrounding tissue image. The third lens 232 may be a lens for generating the left surrounding tissue image of the object 10 and the fourth lens 234 may be a lens for generating the right surrounding tissue image of the object 10. When the endoscope 200 is inserted into the body of the person to be diagnosed, the third lens 232 may be disposed at a left side of the first lens 222 and the fourth lens 234 may be disposed at a right side of the second lens 224. Because the third and fourth lenses 232 and 234 are used to obtain the surrounding tissue image of the object 10 and prevent an organ in the body of the person to be diagnosed from being damaged when the endoscope 200 is inserted into the body of the person to be diagnosed, viewing fields of the third and fourth lenses 232 and 234 may be wide. For example, the viewing fields of the third and fourth lenses 232 and 234 may be greater than 120 degrees. That is, the viewing fields of the third and fourth lenses 232 and 234 may be greater than the viewing fields of the first and second lenses 222 and 224.

Because the depth information is obtained by using the first and second lenses 222 and 224, viewing areas of the first and second lenses 222 and 224 may overlap each other. Because the first lens unit 220 and the second lens unit 230 are used to know relative positions of the object 10 and the surrounding tissue of the object 10, the viewing area of the first lens 222 and the viewing area of the third lens 232 may partially overlap each other, and the viewing area of the second lens 224 and the viewing area of the fourth lens 234 may partially overlap each other. Accordingly, the object information may be generated by using the first and second lenses 222 and 224, and the object image and the surrounding tissue image may be continuously generated by using the first through fourth lenses 222, 224, 232, and 234.

Also, the switching unit 240 may alternately apply light received from the first lens unit 220 and the second lens unit 230 to the photographing unit 210 at predetermined time intervals. The predetermined time intervals may be in units of frames. That is, the switching unit 240 may apply light received from the first lens unit 220 to the photographing unit 210 during odd frames and may apply light received from the second lens unit 230 to the photographing unit 210 during even frames. In detail, the switching unit 240 may include a first switching unit 242 that alternately applies light received from the first and third lenses 222 and 232 to the first photographing unit 212 and a second switching unit 244 that alternately applies light received from the second and fourth lenses 224 and 234 to the second photographing unit 214.

Although the endoscope 200 includes the third and fourth lenses 232 and 234 in order to obtain the surrounding tissue image of the object in FIG. 2, the present embodiment is not limited thereto. Only one of the third and fourth lenses 232 and 234 may be provided in order to obtain only one of the left surrounding tissue image and the right surrounding tissue image of the object 10. For convenience of explanation, a case where both the third and fourth lenses 232 and 234 are provided will now be explained.

As such, because the endoscope 200 may capture not only the object 10, but also the surrounding tissue of the object 10 by using a plurality of photographing elements, the endoscope 200 has a wide viewing field.

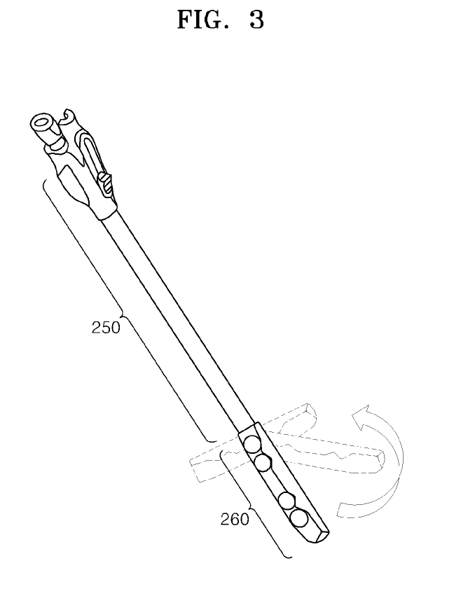

FIG. 3 is a perspective view illustrating the endoscope 200 according to an embodiment of the present disclosure. Referring to FIG. 3, the endoscope 200 includes a first insertion unit 250 adapted to be inserted into the body of the person to be diagnosed, and a second insertion unit 260 that is disposed on a front end of the first insertion unit 250 and allows the first through fourth lenses 222, 224, 232, and 234, the first and second switching units 242 and 244, and the first and second photographing units 212 and 214 to be provided therein. Each of the first and second insertion units 250 and 260 may have a narrow and long cylindrical shape.

The second insertion unit 260 which is disposed on the front end of the first insertion unit 250 may pivot about the front end of the first insertion unit 250 after the endoscope 200 is inserted into the body of the person to be diagnosed, as shown in FIG. 3. The endoscope 200 may include a sliding unit (not shown) that connects the second insertion unit 260 and the first insertion unit 250 and slides toward a lateral end of the second insertion unit 260, and a guide unit (not shown) that is disposed on the lateral end of the second insertion unit 260 and guides the sliding unit.

Before the endoscope 200 is inserted into the body of the person to be diagnosed, the second insertion unit 260 is disposed parallel to the first insertion unit 250 in a longitudinal direction of the first insertion unit 250. In a state where the second insertion unit 260 is disposed parallel to the first insertion unit 250, the endoscope 200 is inserted into the body of the person to be diagnosed. However, when the object 10 and the surrounding tissue of the object 10 is photographed after the endoscope 200 is inserted into the body of the person to be diagnosed, the second insertion unit 260 is disposed perpendicular to the longitudinal direction of the first insertion unit 250 such that the second insertion unit 260 rotates about the front end of the first insertion unit 250 and the sliding unit slides along the guide unit. Even when the second insertion unit 260 is disposed perpendicular to the longitudinal direction of the first insertion unit 250, the second insertion unit 260 and the first insertion unit 250 may be concentric. After photographing is finished, the second insertion unit 260 pivots in the opposite direction, is disposed parallel to the first insertion unit 250, and then is removed from the body of the person to be diagnosed.

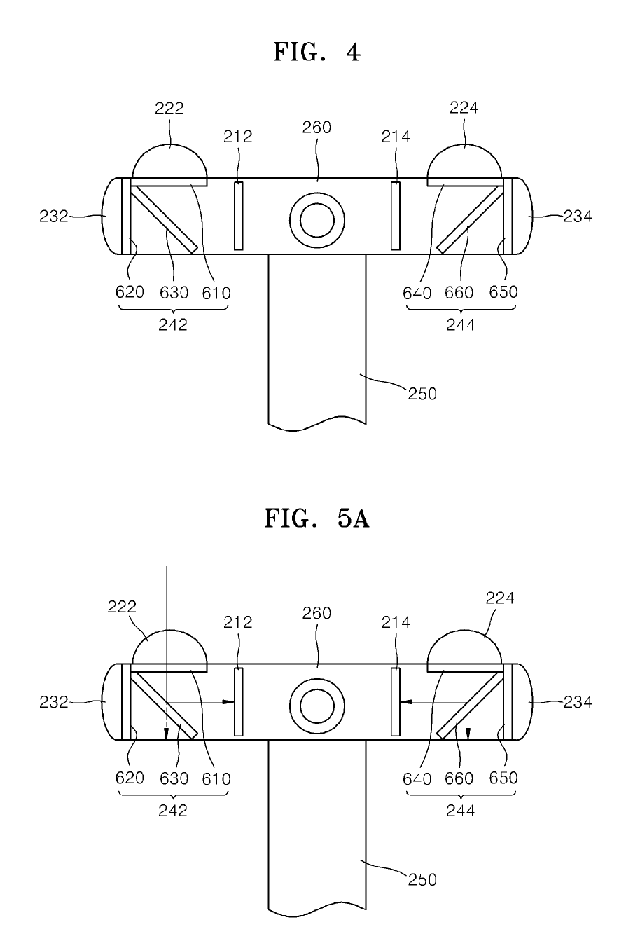

FIG. 4 is a cross-sectional view illustrating an optical arrangement when the endoscope 200 photographs the object 10 and the surrounding tissue of the object 10, according to an embodiment of the present disclosure.

The first and second lenses 222 and 224 may be disposed on the lateral end of the second insertion unit 260 to be parallel to each other, and the third and fourth lenses 232 and 234 may be respectively disposed on a front end and a rear end of the second insertion unit 260. When photographing is performed after the endoscope 200 is inserted into the body of the person to be diagnosed, the second insertion unit 260 pivots counterclockwise and is disposed perpendicular to the first insertion unit 250. Accordingly, as shown in FIG. 4, the first and second lenses 222 and 224 are disposed perpendicular to the first insertion unit 250 to face the object 10, and the third and fourth lenses 232 and 234 are disposed parallel to the first insertion unit 250.

A distance between the first and second lenses 222 and 224 is proportional to resolution of the depth information. That is, as the distance between the first and second lenses 222 and 224 increases, more precise depth information may be obtained. In general, because lenses used to obtain an object image are disposed on a front end of an endoscope, a distance between the lenses may not be greater than a cross-sectional area of the endoscope. However, according to the present embodiment, because the second insertion unit 260 of the endoscope 200 may rotate, the first and second lenses 222 and 224 may be disposed on the lateral end of the second insertion unit 260 and may have large areas without being limited by a cross-sectional area of the endoscope 200. Accordingly, the resolution of the depth information may be increased.

The third lens 232 may be disposed at a left side of the first lens 222 and the fourth lens 234 may be disposed at a right side of the second lens 224, on the both ends of the second insertion unit 260. Accordingly, the third and fourth lenses 232 and 234 may be used to obtain the left surrounding tissue image and the right surrounding tissue image of the object 10, respectively. Also, because the second insertion unit 260 rotates about the first insertion unit 250, upper and lower surrounding tissue images of the object 10 as well as the left and right surrounding tissue images of the object 10 may be obtained.

Although the third and fourth lenses 232 and 234 are disposed on the both ends of the second insertion unit 260, the present embodiment is not limited thereto. The third and fourth lenses 232 and 234 may be disposed on the lateral end of the second insertion unit 260. In this case, the lateral end of the second insertion unit 260 may be curved to have a convex portion and an edge, and the first and second lenses 222 and 224 may be disposed on the convex portion and the third and fourth lenses 232 and 234 may be disposed on the edge.

The first and second photographing units 212 and 214 and the first and second switching units 242 and 244 may be disposed in the second insertion unit 260. The first photographing unit 212 may be disposed on an optical path through which light passing through the first lens 222 and the third lens 232 may be received, and the second photographing unit 214 may be disposed on an optical path through which light passing through the second lens 224 and the fourth lens 234 may be received. The first switching unit 242 may be disposed between the first photographing unit 212 and the first and third lenses 222 and 232, and the second switching unit 244 may be disposed between the second photographing unit 214 and the second and fourth lenses 224 and 234.

The switching unit 240 may include a first shutter unit that opens and closes the first lens unit 220, a second shutter unit that opens and closes the second lens unit 230, and a reflecting unit that transmits part of light incident through the first and second lens units 220 and 230 and reflects the rest. In detail, the first shutter unit may include a first shutter 610 that opens and closes the first lens 222 and a second shutter 640 that opens and closes the second lens 224, the second shutter unit may include a third shutter 620 that opens and closes the third lens 232 and a fourth shutter 650 that opens and closes the fourth lens 234, and the reflecting unit may include a first reflecting unit 630 that applies light incident through the first and third lenses 222 and 232 to the first photographing unit 212 and a second reflecting unit 660 that applies light incident through the second and fourth lenses 224 and 234 to the second photographing unit 214. The first and second reflecting units 630 and 660 may be half-mirrors. The first and second photographing units 212 and 214 may photograph the object 10 or the surrounding tissue of the object 10 according to opening and closing operations of the first through fourth shutters 610, 620, 640, and 650.

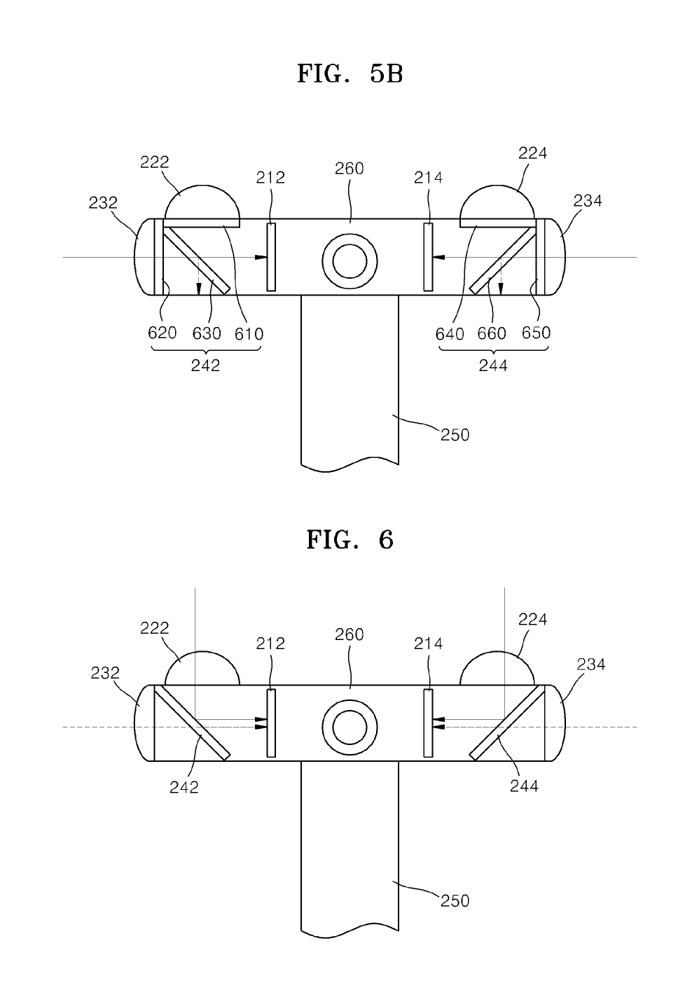

FIG. 5A is a cross-sectional view illustrating an optical path when the endoscope 200 photographs the object 10. FIG. 5B is a cross-sectional view illustrating an optical path when the endoscope 200 photographs the surrounding tissue of the object 10.

Referring to FIG. 5A, when the endoscope 200 photographs the object 10, the third and fourth shutters 620 and 650 are closed and light passing through the third and fourth lenses 232 and 234 is blocked from being incident on the first insertion unit 250. The first and second shutters 610 and 640 are opened and light passing through the first and second lenses 222 and 224 is applied to the first and second reflecting units 630 and 660. The first and second reflecting units 630 and 660 respectively reflect the light incident from the first and second lenses 222 and 224 to the first and second photographing units 212 and 214. Accordingly, the first photographing unit 212 captures the left-eye image of the object 10 and the second photographing unit 214 captures the right-eye image of the object 10.

Referring to FIG. 5B, when the endoscope 200 photographs the surrounding tissue of the object 10, the first and second shutters 610 and 640 are closed and light passing through the first and second lenses 222 and 224 is blocked from being incident on the first insertion unit 250. The third and fourth shutters 620 and 650 are opened and light passing through the third and fourth lenses 232 and 234 is respectively applied to the first and second reflecting units 630 and 660. The first and second reflecting units 630 and 660 transmit the light incident from the third and fourth lenses 232 and 234 to the first and second photographing units 212 and 214. Accordingly, the first photographing unit 212 captures the left surrounding tissue image of the object 10 and the second photographing unit 214 captures the right surrounding tissue image of the object 10.

Light captured by the first and second photographing units 212 and 214 is converted into an electrical signal and the electrical signal is applied to the processor 300 through the second insertion unit 260. That is, because the second insertion unit 260 does not transmit an optical signal to the processor 300, but transmits the electrical signal to the processor 300, the second insertion unit 260 may be easily manufactured.

Also, the switching unit 240 may be an optical modulator that selectively performs transmission or reflection according to an electrical signal. FIG. 6 is a cross-sectional view illustrating an optical arrangement of the endoscope 200, according to an embodiment of the present disclosure.

The optical arrangement of the first and second photographing units 212 and 214, the first through fourth lenses 222, 224, 232, and 234, and the first and second switching units 242 and 244 of FIG. 6 and the optical arrangement of the first and second photographing units 212 and 214, the first through fourth lenses 222, 224, 232, and 234, and the first and second switching units 242 and 244 of FIG. 5 are the same. However, in FIG. 6, the first and second switching units 242 and 242 may be optical modulators that reflect light incident through the first lens unit 220 and transmit light incident through the second lens unit 230 to the first and second photographing units 212 and 214 according to an electrical control signal. Each of the optical modulators may include a liquid crystal device and a reflective polarizer. In detail, the first switching unit 242 may alternately reflect light incident through the first lens 222 and transmit light incident through the third lens 232 according to an electrical control signal, and the second switching unit 244 may alternately reflect light incident through the second lens 224 and transmit light incident through the fourth lens 234 according to an electrical control signal. Because the first and second switching units 242 and 244 are synchronized, the first and second switching units 242 and 244 may alternately perform reflection and transmission.

Accordingly, when an electrical control signal is a reflection signal, light passing through the first and second lenses 222 and 224 are respectively reflected by the first and second switching units 242 and 244 to the first and second photographing units 212 and 214. The first photographing unit 212 captures the left-eye image of the object 10 and the second photographing unit 214 captures the right-eye image of the object 10. When an electrical control signal is a transmission signal, light passing through the third and fourth lenses 232 and 234 are respectively transmitted through the first and second switching units 242 and 244 to the first and second photographing units 212 and 214. The first photographing unit 212 captures the left surrounding tissue image of the object 10 and the second photographing unit 214 captures the right surrounding tissue image of the object 10. Light captured by the first and second photographing units 212 and 214 is converted into an electrical signal, and the electrical signal is applied to the processor 300 through the second insertion unit 260.

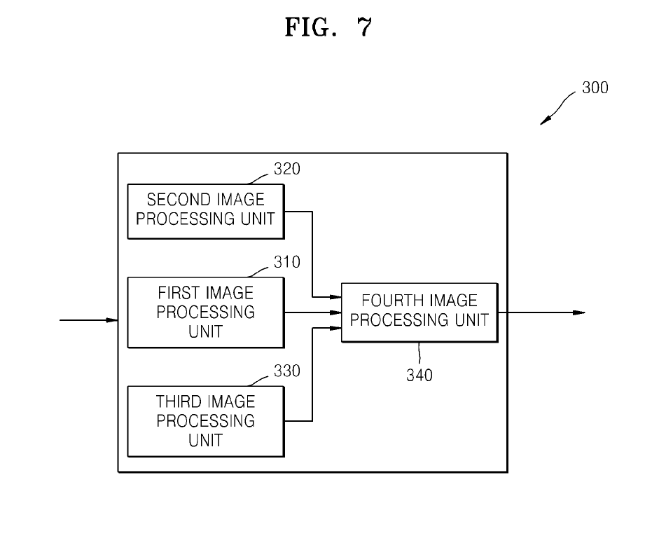

An operation of the processor 300 for generating a panoramic image will now be explained in detail. FIG. 7 is a block diagram illustrating the processor 300 according to an embodiment of the present disclosure.

The processor 300 receives an image signal of the object 10 from the endoscope 200, and generates a panoramic image by combining the object image of the object 10 and the surrounding tissue image of the object 10. For example, referring to FIG. 7, the processor 300 may include a first image processing unit 310 that calculates the depth information of the object 10 and generates the object image including the depth information, a second image processing unit 320 that generates the left surrounding tissue image of the object 10, a third image processing unit 330 that generates the right surrounding tissue image of the object 10, and a fourth image processing unit 340 that generates a panoramic image by combining the object image, the left surrounding tissue image of the object 10, and the right surrounding tissue image of the object 10.

The first image processing unit 310 receives a left-eye image signal of the object 10 from the first photographing unit 212, and receives a right-eye image signal of the object 10 from the second photographing unit 214. The first image processing unit 310 obtains the depth information of the object 10 from the left-eye image signal and the right-eye image signal. The first image processing unit 310 may obtain the depth information of the entire object 10 and generate a three-dimensional (3D) stereoscopic image, or may obtain the depth information of a specific area of the object 10 and generate the object image including the depth information. A method of generating the object image from an image signal from the photographing unit 210 is well known in the technical field related to 3D image processing, and thus, a detailed explanation thereof will not be given.

The second image processing unit 320 receives a left surrounding tissue image signal of the object 10 from the first photographing unit 212 and generates the left surrounding tissue image, and the third image processing unit 330 receives a right surrounding tissue image signal of the object 10 from the second photographing unit 214 and generates the right surrounding tissue image. The left surrounding tissue image and the right surrounding tissue image may be two-dimensional (2D) images.

The fourth image processing unit 340 generates a panoramic image by combining the left surrounding tissue image, the object image, and the right surrounding tissue image. In order to generate the panoramic image, the fourth image processing unit 340 combines the left surrounding tissue image and the right surrounding tissue image based on the object image to display the object image on a common area.

The object image is an image captured during odd frames, and the left surrounding tissue image and the right surrounding tissue image are images captured during even frames. Accordingly, the object image and the left and right surrounding tissue images are not images captured at the same time. However, because an environment in the body of the person to be diagnosed does not change quickly in units of frames, the panoramic image obtained by combining the object image, the left surrounding tissue image, and the right surrounding tissue image may provide sufficient information about the environment in the body of the person to be diagnosed to a user. Accordingly, because the user may perform an operation by using the object image and insert the endoscope 200 into the body cavity while seeing the left and right surrounding tissue images, the operation may be more safely performed.

Because the display device 400 of the endoscope system 100 displays the panoramic image, the endoscope system 100 may provide information about the surrounding tissue of the object 10 as well as the object 10. Each of the processor 300 and the display device 400 may include a computer.

Because the present disclosure provides a panoramic image obtained by combining an object image including depth information and a surrounding tissue image, more convenience may be provided to an endoscope user.

The above-described embodiments may be recorded in computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter. The above-described devices may be configured to act as one or more software modules in order to perform the operations of the above-described embodiments, or vice versa.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An endoscope comprising:
a first lens unit that collects light corresponding to an object image, which is an image comprising depth information of an object;
a second lens unit that collects light corresponding to a surrounding tissue image, which is an image of a surrounding tissue of the object; and
a switching unit that alternately applies light received from the first lens unit and the second lens unit to a photographing unit; and
a photographing unit that alternately captures the light from the switching unit.

2. The endoscope of claim 1, wherein a viewing field of the second lens unit is greater than a viewing field of the first lens unit.

3. The endoscope of claim 2, wherein a viewing area of the second lens unit and a viewing area of the first lens unit partially overlap each other.

4. The endoscope of claim 1, wherein the switching unit applies light received from the first lens unit to the photographing unit during a first time interval, and applies light received from the second lens unit to the photographing unit during a second time interval.

5. The endoscope of claim 1, wherein the switching unit comprises:
a first shutter unit that opens and closes the first lens unit;
a second shutter unit that opens and closes the second lens unit; and
a reflecting unit that transmits part of light incident from the first and second lens units and reflects the rest.

6. The endoscope of claim 5, wherein the reflecting unit reflects light incident from the first lens unit to the photographing unit, and transmits light incident from the second lens unit to the photographing unit.

7. The endoscope of claim 5, wherein the reflecting unit comprises a half-mirror.

8. The endoscope of claim 1, wherein the switching unit is an optical modulator that reflects light incident from the first lens unit, and transmits light incident from the second lens unit.

9. The endoscope of claim 8, wherein the optical modulator alternately reflects and transmits light incident according to an electrical control signal.

10. The endoscope of claim 1, wherein the surrounding tissue image is a two-dimensional (2D) image.

11. The endoscope of claim 1, further comprising:
a first insertion unit adapted to be inserted into a body of a person to be diagnosed; and
a second insertion unit that is disposed on a front end of the first insertion unit and on which the photographing unit, the first lens unit, the second lens unit, and the switching unit are provided.

12. The endoscope of claim 11, wherein the second insertion unit is pivotable about the front end of the first insertion unit.

13. The endoscope of claim 11, wherein the second lens unit is disposed to face in a longitudinal direction of the first insertion unit before the second insertion unit is inserted into the body of the person to be diagnosed, and is disposed to face perpendicular to the longitudinal direction of the first insertion unit after the second insertion unit is inserted into the body of the person to be diagnosed.

14. The endoscope of claim 11, wherein the first lens unit is disposed to face perpendicular to a longitudinal direction of the first insertion unit before the second insertion unit is inserted into the body of the person to be diagnosed, and is disposed to face in the longitudinal direction of the first insertion unit after the second insertion unit is inserted into the body of the person to be diagnosed.

15. The endoscope of claim 1, wherein the photographing unit comprises:
a first photographing unit that generates a left-eye image of the object; and
a second photographing unit that generates a right-eye image of the object.

16. The endoscope of claim 15, wherein the first lens unit comprises:
a first lens that generates the left-eye image of the object; and
a second lens that generates the right-eye image of the object.

17. The endoscope of claim 15, wherein the second lens unit comprises:
a third lens that generates a left surrounding tissue image of the object; and
a fourth lens that generates a right surrounding tissue image of the object.

18. The endoscope of claim 17, wherein the first photographing unit captures the left surrounding tissue image and the second photographing unit captures the right surrounding tissue image of the object.

19. An endoscope system comprising:
an endoscope comprising:
a photographing unit that alternately photographs an object and a surrounding tissue of the object;
a first lens unit that collects light corresponding to an object image which is an image comprising depth information of the object;
a second lens unit that collects light corresponding to a surrounding tissue image which is an image of the surrounding tissue of the object; and
a switching unit that alternately applies light received from the first lens unit and the second lens unit to the photographing unit; and
a processor that generates a panoramic image by combining the object image and the surrounding tissue image.

20. The endoscope system of claim 19, further comprising a display device that displays the panoramic image.

21. A method of providing a panoramic view of an inside of a body cavity, the method comprising:
inserting an endoscope, comprising an extension portion and a rotatable end portion, into the body cavity such that the end portion is inside the body cavity;
rotating the end portion inside the body cavity, such that the rotated end portion is perpendicular to the extension portion;
capturing, using the end portion, a first and second image of the body cavity in the direction of insertion;
capturing, using the end portion, a third and fourth image of the body cavity perpendicular to the direction of insertion;
combining the first, second, third, and fourth images as a panoramic image of the body cavity.

22. The method of claim 21, wherein the first image and the second image are captured from opposite ends of the end portion, and the third image and the fourth image are captured from opposite ends of the end portion.

23. The method of claim 22, wherein the panoramic image comprises a three-dimensional (3D) stereoscopic image.
